(19) United States
(12) Patent Application Publication
(10) Pub. No.: US 2008/0241843 A1
(43) Pub. Date: Oct. 2, 2008

(54) Single-cell analysis systems, methods of counting molecules in a single-cell, cylindrical fluorescence detection systems

(75) Inventors: Richard N. Zare, Stanford, CA (US); Bo Huang, Cambridge, MA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: Stanford University, Palo Alto, CA (US)

(21) Appl. No.: 12/004,874

(22) Filed: Dec. 21, 2007

Related U.S. Application Data
(60) Provisional application No. 60/876,422, filed on Dec. 21, 2006.

(51) Int. Cl. C12Q 1/68; C12M 1/00; G01J 1/58; C12Q 1/02

(52) U.S. Cl. 435/6; 435/287.1; 435/288.7; 435/29; 250/458.1

(57) ABSTRACT

Embodiments of the present disclosure provide for single-cell analysis systems, methods of detecting target components in a single cell, cylindrical fluorescence detection systems, and the like.

### CROSS-REFERENCE TO RELATED APPLICATION

This application claims priority to U.S. provisional applications entitled, “SINGLE-CELL ANALYSIS SYSTEMS AND METHODS OF COUNTING MOLECULES IN A SINGLE-CELL,” having Ser. No. 60/876,422, filed on Dec. 21, 2006, which is entirely incorporated herein by reference.

### FEDERAL SPONSORSHIP

This invention was made with Government support under Contract/Grant No. BES-0508531, awarded by the National Science Foundation. The Government has certain rights in this invention.

### BACKGROUND

Single-cell analysis has become a highly attractive tool for investigating cellular contents. Unlike conventional methods that are performed with large cell populations, this technology avoids the loss of information associated with ensemble averaging. Recently, several researchers have reported on methods that can quantify specific proteins inside a single cell via means of integrated fluorescence and in one instance with spatial resolution. These approaches are limited to those special cases where the environment of the cell does not cause changes in the fluorescence of the reporter molecule and where quenching and endogenous fluorescence do not interfere with the measurements. Moreover, these techniques restrict viewing to one or perhaps a few species at a time.

Low-copy-number proteins (present at less than a few thousand molecules per cell) play an important role in cell functioning, including signaling and the regulation of gene expression. Without amplification procedures, their abundance is far below the sensitivity limits of conventional protein analysis methods, such as ELISA and mass spectroscopy.

Therefore, there is a need in the art to analyze the biomolecules present in a single cell, particularly those present in low concentrations.

### SUMMARY

Embodiments of the present disclosure provide for single-cell analysis systems, methods of detecting target components in a single cell, cylindrical fluorescence detection systems, and the like.

One exemplary single-cell analysis system, among others, includes: a cell manipulation system, wherein the cell manipulation system includes a reaction chamber, a cell suspension separation system, a lysis system, and a labeling system, wherein the reaction chamber is interfaced with the cell suspension separation system, the lysis system, and the labeling system through a fluid exchange control system; a separation system, wherein the reaction chamber is interfaced with the separation system through the fluid exchange control system; and a detection system, wherein the detection system is interfaced with the separation system.

One exemplary method of detecting target components in a single cell, among others, includes: isolating a single cell from a cell suspension including a plurality of cells; lysing the cell to release the components in the cell; separating the target components from the other components released from the cell; and detecting the target components.

One exemplary cylindrical fluorescence detection system, among others, includes: a laser system capable of emitting a laser beam; and a cylindrical optic system, wherein cylindrical optic system is configured to receive the laser beam, wherein the cylindrical optic system includes two lenses, wherein the first lens is non-circularly symmetric with respect to the direction of the laser beam, wherein the first lens receives the laser beam, wherein the first lens is configured to focus the laser beam to form a line at a back focal plane of the second lens, wherein the first lens is configured to direct the focused laser beam to the second lens, wherein the second lens is configured to collimate the laser beam received from the first lens in the direction perpendicular to a channel length of a channel, wherein the collimated laser beam has a width that extends the width of the channel, wherein the second lens is configured to focus the laser beam received from the first lens in the direction parallel to the channel length of the channel.

These embodiments, uses of these embodiments, and other uses, features and advantages of the present disclosure, will become more apparent to those of ordinary skill in the relevant art when the following detailed description of the preferred embodiments is read in conjunction with the appended figures.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

The patent or application file contains at least one drawing executed in color. Copies of this patent or patent application publication with color drawing(s) will be provided by the Office upon request and payment of the necessary fee.

FIG. 1 illustrates a block diagram of an exemplary embodiment of a single-cell analysis system.

FIG. 2 is a flow chart illustrating an embodiment of a method of detecting target components in a single cell.

FIG. 3 is a flow chart illustrating another embodiment of a method of detecting target components in a single cell.

FIG. 4A is a schematic illustration of the excitation laser focused by the microscope objective and the dimension of the molecule counting channel.

FIG. 4B illustrates a frame from the CCD images of A647-SA flowing across the molecule counting section and the identification results.

FIG. 4C illustrates a CE separation of 100 nM A647-SA.

FIG. 4D illustrates a molecule counting of 73 μM A647-SA with the “slow-flow” method, showing the number of identified molecules in each frame of image and the average molecule count rate in one-second time bins. The injection plug size is 35 μL.

FIGS. 5A-5C illustrate Synechococcus sp. PCC 7942 grown in nitrogen-replete culture medium (+N) and nitrogen-depleted medium (—N). In particular, FIG. 5A illustrates a photograph of cell cultures in replete (left) and depleted medium (right).

FIG. 5B illustrates an absorption spectrum of cells, normalized by the absorption at 750 nm (proportional to the cell density). Changes in PBS and chlorophyll (chl) absorption are marked. FIG. 5C illustrates an electropherogram of the cell lysates. The —N lysate is 12 times as concentrated as the +N lysate to have a similar fluorescence signal level.

FIGS. 6A-6D illustrate an embodiment of a design and the operation of the single-cell analysis chip. In particular, FIG. 6A illustrates a photograph of the chip. The inset shows the cell manipulation region viewed through a microscope (scale bar 300 μm). FIG. 6B illustrates a schematic chip layout (dimensions in microns). FIG. 6C illustrates the operation procedure of cell capturing, lysis and analysis. FIG. 6D illustrates fluorescence images of a Synechoccocus cell captured in the reaction chamber at different times during the lysis procedure.

FIGS. 7A-7C illustrate the results of single-cell analysis. FIG. 7A illustrates single-cell electropherograms of three +N cells. The curves are vertically shifted for clarity. Small shifts among them can be attributed to the slight difference in the separation channel length. FIG. 7B illustrates the molecule counting results of three −N cells. FIG. 7C illustrates the molecule number distribution of twelve −N cells. The lysing and counting efficiencies are corrected individually. Results from the three cells in FIG. 7B are marked. Red lines show the result of least square linear fitting. The inset shows cell (a), which is excluded from the fitting because otherwise its value would dominate the fit.

FIGS. 8A and 8B illustrates the creation of the detection curtain. In particular, FIG. 8A illustrates the layout of the cylindrical optics. FIG. 8B illustrates the z-dependence of detected fluorescence from a glass surface coated with Atto 565 labeled streptavidin (Sigma-Aldrich). The fluorescence intensity for the wide-field configuration is measured by averaging a 20 pixel×20 pixel area at the center of the view field; the fluorescence intensity for the cylindrical configuration is measured by averaging 20 continuous pixels horizontally aligned at the middle of the focus line; and the fluorescence intensity for the confocal configuration is characterized by the intensity of the pixel at the focal point. The range of z that is covered by the molecule counting channel is marked by green dashed lines.

FIGS. 9A and 9B illustrate the image analysis procedure for the separation and counting of A647-SA molecules. In each panel, the upper part is the original image recorded by the CCD camera, the lower part is the image after Fourier filtering, and the colored line between them shows the cross-section of the Fourier filtered image along the detection curtain. In the lower parts, colored regions mark the pixels that are brighter than the threshold. The regions not identified as valid molecule counts appear blue. In particular, FIG. 9A illustrates the improvement in identification when overlapped fluorescent spots can be split (lower panel). FIG. 9B illustrates when one molecule is imaged in two consecutive frames, the fluorescent spot has the same x position in both frames.

FIGS. 10A-10D illustrates the analysis of A647-SA in a double-T chip. In particular, FIG. 10A illustrates the layout of the “double-T” chip for A647-SA separation. FIG. 10B illustrates the fluorescence images of the double-T junction when separation starts. Dotted lines show the outline of the channels. Timing starts when the voltage set applied to the chip is switched from loading (1=1000 V, 2=700 V, 3=0 V, and 4=1000 V) to separation (1=700 V, 2=1000 V, 3=700 V, and 4=0 V). Arrows indicate the flow direction. FIG. 10C illustrates the CE separation of 100 nM A647-SA. FIG. 10D illustrates the molecule counting of 73 μM A647-SA by lowering the voltage to 1/10 of the ordinary values when the analyte passes the detection curtain, showing the number of identified molecules in each frame of image (black bars) and the average molecule count rate in one-second time bins (red line).

FIGS. 11A-11B illustrate the dependence of molecule counts on the threshold. FIG. 11A illustrates the counting of A647-SA molecules. The error bars in A647-SA counts are the standard deviations of seventeen measurements, and those in blank counts are the standard deviations in three measurements. FIG. 11B illustrates the molecule counts in peak 2 in cell (c) of FIG. 7B. The blank control is measured in the same chip with no separation voltage applied.

FIG. 12 illustrates the electrophoretic analysis of SF9 lysate reacted with excess amount of Cy5-M1. The x scale is converted to the migration velocity, which corresponds to the displacement along the separation channel of different species at a certain time, so that the integral reflects the total amount of separated analytes.

FIG. 13 illustrates the analysis of individual cyanobacteria cells. FIG. 13A illustrates the operation procedure of cell capturing, lysis and analysis. FIG. 13B illustrates the fluorescence images of a Synechoccocus cell captured in the reaction chamber at different times during the lysis procedure.

### DETAILED DESCRIPTION

Embodiments of the present disclosure will employ, unless otherwise indicated, techniques of separation (e.g., chromatography, electrophoresis, and the like), synthetic chemistry, biochemistry, molecular biology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by volume, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### DEFINITIONS

The term “polypeptides” includes proteins and fragments thereof. Polypeptides are disclosed herein as amino acid residue sequences. Those sequences are written left to right in the direction from the amino to the carboxy terminus. The amino acid residue sequences include, but are not limited to, Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (H is, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

In addition, the polypeptide can include non-standard and/or non-naturally occurring amino acids, as well as other amino acids that may be found in phosphorylated and/or glycosylated proteins in organisms such as, but not limited to, animals, plants, insects, protists, fungi, bacteria, algae, single-cell organisms, and the like. The non-standard amino acids include, but are not limited to, selenocysteine, pyrrolysine, gamma-aminobutyric acid, carnitine, ornithine, citrulline, homocysteine, hydroxyproline, hydroxylysine, sarcosine, and the like. The non-naturally occurring amino acids include, but are not limited to, trans-3-methylproline, 2,4-methanoproline, cis-4-hydroxyproline, trans-4-hydroxyproline, N-methyl-glycine, allo-threonine, methylthreonine, hydroxy-ethylcysteine, hydroxyethylhomocysteine, nitro-glutamine, homoglutamine, pipecolic acid, thiazolidine carboxylic acid, dehydroproline, 3- and 4-methylproline, 3,3-dimethylproline, tert-leucine, norvaline, 2-azaphenylalanine, 3-azaphenylalanine, 4-azaphenylalanine, and 4-fluorophenylalanine.

As used herein, the term “polynucleotide” generally refers to any polyribonucleotide or polydeoxyribonucleotide, which may be unmodified RNA or DNA or modified RNA or DNA. Thus, for instance, polynucleotides as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. The terms “nucleic acid,” “nucleic acid sequence,” or “oligonucleotide” also encompasses a polynucleotide as defined above.

In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide.

As used herein, the term polynucleotide includes DNAs or RNAs as described above that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein.

Representative fluorescent compounds (fluorophores) can include, but are not limited to, sgGFP, sgBFP, BFP blue-shifted GFP (Y66H), Blue Fluorescent Protein, CFP—Cyan Fluorescent Protein, Cyan GFP, DsRed, monomeric RFP, EBFP, ECFP, EGFP, GFP (S65T), GFP red shifted (rsGFP), GFP wild type, non-UV excitation (wtGFP), GFP wild type, UV excitation (wtGFP), GFPuv, HcRed, rsGFP, Sapphire GFP, sgBFP™, sgBFP™ (super glow BFP), sgGFP™, sgGFP™ (super glow GFP), wt GFP, Yellow GFP, YFP, semiconductor nanoparticles (e.g., quantum dots, Raman nanoparticles) or combinations thereof.

Other representative fluorescent compounds (fluorophores) can include, but are not limited to: 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-FAM (5-Carboxyfluorescein); 5-HAT (Hydroxy Tryptamine); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Aequorin (Photoprotein); AFPs-AutoFluorescent Protein-(Quantum Biotechnologies); Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; AMCA (Aminomethylcoumarin); AMCA-X; Aminoactinomycin D; Aminocoumarin; Aminomethylcoumarin (AMCA); Anilin Blue; Anthrocyl stearate; APC (Allophycocyanin); APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; Bimane; Bisbenzamide; Bisbenzimide (Hoechst); bis-BTC; Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy FI; Bodipy FL ATP; Bodipy FI-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson™; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF₂ (GeneBlazer); CFDA; Chlorophyll; Chromomycin A; Chromomycin A; CL-NERF; CMFDA; Coumarin Phalloidin; C-phycocyanine; CPM Methylcoumarin; CTC; CTC Formazan; Cy2™; Cy3.18; Cy3.5™; Cy3™; Cy5.18; Cy5.5™; Cy5™; Cy7™; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′ DCFDA; DCFH (Dichlorodihydrofluorescein Diacetate); DDAO; DHR (Dihydrorhodamine 123); Di-4-ANEPPS; Di-8-ANEPPS (non-ratio); DiA (4-Di-16-ASP); Dichlorodihydrofluorescein Diacetate (DCFH); DiD-Lipophilic Tracer; DiD (DiIC18(5)); DIDS; Dihydrorhodamine 123 (DHR); Dil (DiIC18(3)); Dinitrophenol; DiO (DiOC18(3)); DiR; DiR (DilC18(7)); DM-NERF (high pH); DNP; Dopamine; DTAF; DY-630-NHS; DY-635-NHS; ELF 97; Eosin; Erythrosin; Erythrosin ITC; Ethidium Bromide; Ethidium homodimer-1 (EthD-1); Euchrysin; EukoLight; Europium (III) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; Fluor X; FM 1-43™; FM 4-46; Fura Red™ (high pH); Fura Red™/Fluo-3; Fura-2; Fura-2/BCECF; Genacryl Brilliant Red B; Genacryl Brilliant Yellow 10 GF; Genacryl Pink 3G; Genacryl Yellow 5GF; GeneBlazer (CCF₂); Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1, low calcium; Indodicarbocyanine (DiD); Indotricarbocyanine (DiR); Intrawhite Cf; JC-1; JO-JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxadidole; Noradrenaline; Nuclear Fast Red; Nuclear Yellow; Nylosan Brilliant lavin E8G; Oregon Green; Oregon Green 488-X; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed [Red 613]; Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-PRO-3; Primuline; Procion Yellow; Propidium lodid (PI); PyMPO; Pyrene; Pyronine; Pyronine B; Pyrozal Brilliant Flavin 7GF; QSY 7; Quinacrine Mustard; Red 613 [PE-TexasRed]; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycocyanine; R-phycoerythrin (PE); S65A; S65C; S65L; S65T; SBFI; Serotonin; Sevron Brilliant Red 2B; Sevron Brilliant Red 4G; Sevron Brilliant Red B; Sevron Orange; Sevron Yellow L; SITS; SITS (Primuline); SITS (Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3-sulfopropyl)quinolinium); Stilbene; Sulphorhodamine B can C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC TetramethylRodaminelsoThioCyanate; True Blue; TruRed; Ultralite; Uranine B; Uvitex SFC; WW 781; X-Rhodamine; XRITC; Xylene Orange; Y66F; Y66H; Y66W; YO-PRO-1; YO-PRO-3; YOYO-1; YOYO-3, Sybr Green, Thiazole orange (interchelating dyes), or combinations thereof.

### General Discussion

Embodiments of the present disclosure include single-cell analysis systems and methods of measuring target components (e.g., biomolecules such as, but not limited to, polypeptides, polynucleotides, small molecules, and the like) in a single cell. Embodiments of the present disclosure can be used to isolate a single cell from a cell suspension and release the components inside the cell, and optionally label select components (e.g., target components). The released components are separated using a separation technique and then detected using a detection system (e.g., single molecule detection system).

One advantage of the present disclosure is the ability to quantify one or more target components that cannot be distinguished by their fluorescence properties alone. In addition, low-copy-number proteins present in a cell can be detected using embodiments of the present disclosure. Also, analyzing the components of a single cell can reveal information that would otherwise be hidden by analyzing the components of many cells at the same time. They include mutations and response to various stresses, such as oxidative stress, temperature stress, radiation stress, combinations thereof, unsynchonized behavior in a cell population; rare types of cells in a large biological sample, and the like. Additional advantages and details regarding embodiment of the present disclosure are described in Example 1.

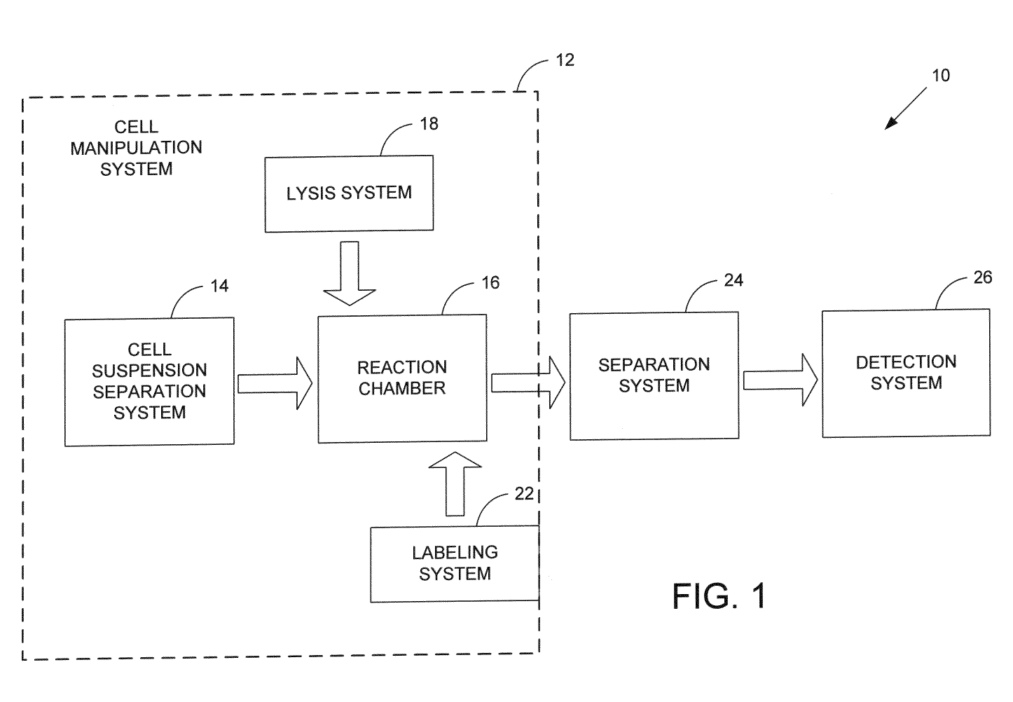

FIG. 1 illustrates a block diagram of an exemplary embodiment of a single-cell analysis system 10. The single-cell analysis system 10 includes, but is not limited to, a cell manipulation system 12, a separation system 24, and a detection system 26, all of which are in direct and/or indirect fluidic communication with each other. The cell manipulation system 12 includes, but is not limited to, a reaction chamber 16, a cell suspension separation system 14, a lysis system 18, and a labeling system 22. The lysis system 18 can include one or more methods of lysing the cells such as, but not limited to, chemical lysing, pressure (e.g., shock wave) lysing, laser lysing, mechanical lysing, and the like, and includes the appropriate system components and/or reagents to achieve lysis. The reaction chamber 16 is interfaced (e.g., in fluidic communication) to each of the cell suspension separation system 14, the lysis system 18, and the labeling system 22. The dimensions of the components of the single-cell analysis system 10 are on the microscale. Another exemplar embodiment of a configuration of the cell manipulation system is described in Example 1.

In an embodiment, the lysis system and the labeling system can be merged into a single system. In addition, the reaction chamber 16 can be interfaced with additional systems such as, but not limited to, buffer reagent systems (e.g., including one or more buffers), rinse systems (e.g., including one or more rinsing reagents), and the like. It should also be noted that the reaction chamber 16 is interfaced with the separation system 24.

The cell suspension separation system 14 includes, but is not limited to, a microfluidic valve system (e.g., a two- or three-state valve design) that separates a single cell from a cell suspension. The cell suspension separation system 14 includes one or more chambers, flow channels, and the reaction chamber 16, so that one or more cells can be flowed into and out of portions of the cell suspension separation system 14. The flow within chambers, the flow channels, and the reaction chamber 16 can be controlled using one or more two-state and/or three-state valves. The flow of the cells can be conducted in a manner to separate one cell from the other cells, where a single cell remains in the reaction chamber 16. Additional details regarding the cell suspension separation system 14 are described in Example 1.

After a single cell has been separated from a cell suspension using the cell suspension separation system 14, the single cell can be lysed (e.g., using known lysing agents) in reaction chamber 16 to release the cell contents or components, which can include, but is not limited to, polypeptides, polynucleotides, fragments thereof, and the like. The cell contents may include one or more types of target components (e.g., one or more target polypeptides and/or biomolecules). If the target component needs to be labeled for detection purposes, then the target component can be labeled while in the reaction chamber 16, or alternatively in a chamber in fluidic communication with the reaction chamber 16. The target components can be labeled using tags such as, but not limited to, fluorescent tags, luminescent molecules (such as, but not limited to, luminol), bioluminescent molecules (such as, but not limited to, luciferases, luciferins, and aequorins), and the like. For example, a fluorescent tag can be attached to one or more types of target components so that the labeled target components can be detected using a fluorescent detection system. In an embodiment, the labeling can be performed prior to the cell lysis. Additional details regarding the reaction chamber 16 are described in Example 1.

Subsequently, the target components (e.g., labeled and/or unlabeled target component) are separated from the other components released from the cell using a separation system 24, which is interfaced with the reaction chamber 16. The separation system 24 can include, but is not limited to, an electrophoresis system (e.g., capillary electrophoresis), a chromatography system (e.g., liquid chromatography), combinations thereof, and the like. The target components can include, but are not limited to, target amino acids, target small molecules, target cell organelles, target polypeptides, a target polynucleotides, target polypeptide-polynucleotide complexes, and the like.

After separation, the separated target components are detected in the detection system 26. The detection system 26 used depends, at least in part, upon the labeling tag employed. In an embodiment, the target components are detected using a single molecule detection system 26 that can detect, for example, fluorescently labeled target components. The fluorescently labeled target components are detected (e.g., counted) by monitoring the number of fluorescent bursts generated as the components flow through a channel having a small detection volume that is in the path of a light source, or by measuring the total fluorescence signal emitted from the detection volume, which is proportional to the concentration of the fluorescent analyte. Analytes labeled with luminescent or bioluminescent probes can be detected in similar ways. In another embodiment, bask-scattering interferometry (See, Science, 317, 1732 (2007), which is incorporated herein by reference), could be used to detect the change in index of refraction; while in another embodiment, thermal lens spectrometry (See, Lab on a Chip, 6, 127-130 (2006), which is incorporated herein by reference), could be used to detect the change in light absorbance.

In particular, the detection system 26 is a cylindrical fluorescence detection system. The cylindrical fluorescence detection system includes cylindrical optics to widen the excitation laser focus. An excitation laser beam is focused by a non-circularly symmetric lens with respect to the direction of the laser beam (e.g., a cylindrical lens) of the cylindrical fluorescence detection system to form a line at the back focal plane of a second lens (e.g., a microscope objective). When the laser beam emerges from the second lens, it is collimated in the direction perpendicular to the channel length, thus capable of covering a channel width of tens of microns (e.g., 1 to 100 microns), which is sufficient to illuminate channels used in fluorescence detection systems. The channel has a width of about 1 to 100 microns and a height of about 0.5 to 10 microns. In the other direction, the laser is still tightly focused by the spherical lens to minimize the fluorescence background from out-of-focus excitation. The channel height needs to fit the z dimension of the excitation focus, which is about 0.5 to 10 μm or about 2 μm depending on the numerical aperture of the second lens. Using a microscope objective with a numerical aperture >1 as the second lens allows the detection efficiency to be high enough so that fluorescence signal from individual molecules can be observed. The rectangular, curtain-shaped detection region across the channel allows labeled target components to be detected as they pass through the detection region.

In an embodiment, the burst of fluorescence from molecules or particles that pass through the curtain is recorded by an intensified CCD camera and counted. This count gives a direct quantification of the total number of target molecules or particles being analyzed. The ability of being able to count individual molecules with high efficiency (enabled by the cylindrical fluorescence detection system) provides sufficient sensitivity for detecting analytes with extremely low amount, for example, low-copy-number proteins released from one cell.

In another embodiment, the fluorescence emission from the analyte passing through the detection curtain is recorded by a photomultiplier tube. A slit is put in front of the photomultiplier tube with its position matching the image of the detection curtain so that out-of-focus background can be rejected. The slit has a length of about 1 to 10 mm and a width of about 20 to 100 microns. The slit reduces the background noise so that detection of the analyte is enhanced.

The concentration of the analyte can be obtained from the intensity of the recorded fluorescence signal. Additional details regarding the detection system are described in more detail in Example 1.

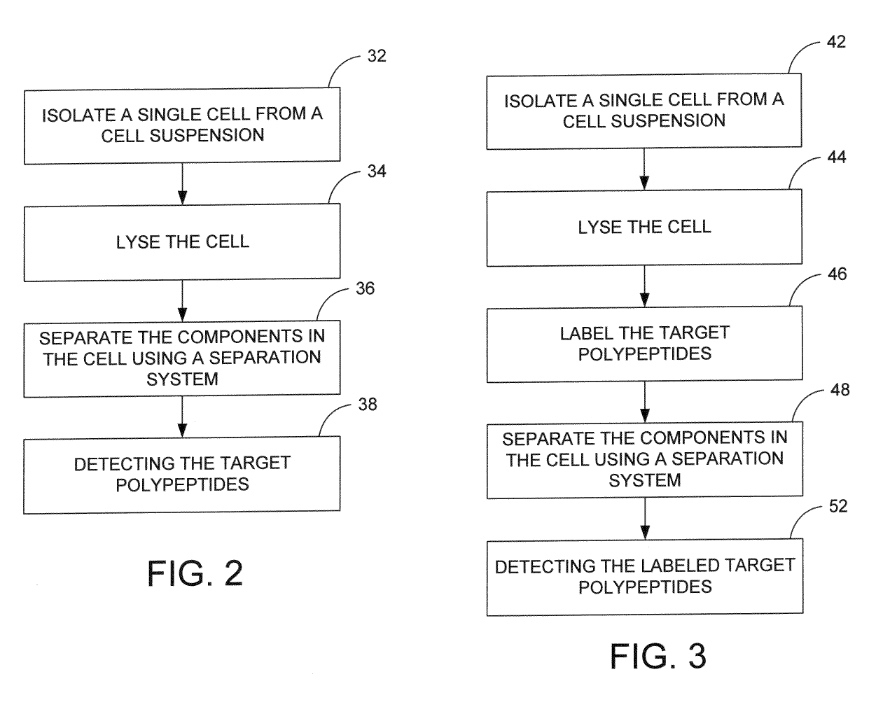

In addition, embodiments of the present disclosure include methods of detecting target components in a single cell, as shown in FIG. 2. In an embodiment, the method includes isolating a single cell from a cell suspension (block 32). For example, an embodiment of the single-cell analysis system can be used to isolate the single cell from a cell suspension. The single cell can be lysed (block 34), which releases the components present in the single cell. The components can be separated (block 36) using a separation system so that the target components can be subsequently detected. In an embodiment, the target components inherently include characteristics (e.g., fluorescent) that enable detection of the target components without the need to attach an external label that can be detected by the detection system. The target components can be detected using one or more detection techniques (block 38).

Another embodiment of the present disclosure includes methods of detecting target components in a single cell, as shown in FIG. 3. In an embodiment, the method includes isolating a single cell from a cell suspension (block 42) (e.g., the single-cell analysis system). The single cell can be lysed (block 44), which releases the components present in the single cell. One or more labeling tags can be introduced to the released components to label one or more target components (block 46). The label tags can be specific for a particular target component so that different types of target components can be detected and identified. In an embodiment, the labeling tag is a fluorescent tag. The components can be separated using a separation system (block 48) so that the target components can be detected. The target components can be detected using one or more detection techniques (block 52). For example, a cylindrical fluorescence detection system can be used to detect the target components. The detection can be performed by either measuring fluorescence intensity or by single-molecule counting.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified. In addition, the phrase “about ‘x’ to ‘y’” includes “about ‘x’ to about ‘y’”.

The above discussion is meant to be illustrative of the principles and various embodiments of the present disclosure. Numerous variations and modifications will become apparent to those skilled in the art once the above disclosure is fully appreciated. It is intended that the following claims be interpreted to embrace all such variations and modifications.

### EXAMPLES

Now having described the embodiments of the disclosure, in general, the example describes some additional embodiments. While embodiments of present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

### Introduction

We have designed a microfluidic device in which we can manipulate, lyse, separate, and quantify the protein contents of a single cell using single-molecule fluorescence detection. The use of cylindrical optics enables high-efficiency single-molecule counting in a micrometer-sized channel. We use this microfluidic device to analyze phycobiliprotein content and the aggregation states of these pigment proteins in individual cyanobacterial cells (Synechococcus sp. PCC 7942) grown under nitrogen-replete or nitrogen-depleted conditions. In the lafter case, we have examined the copy number of phycobiliprotein complexes and their distribution within populations of cells, demonstrating marked differences in the levels of specific complexes in the individuals of a cyanobacterial population experiencing nitrogen deprivation.

### Discussion:

Our solution to the issues noted above include the use of highly sensitive single-molecule fluorescence detection, which has been applied to counting DNA or protein molecules in sheathed flows, capillaries, and microfluidic channels (Anal. Chem. 65, 849 (1993), Anal. Chem. 68, 690 (1996), and Electrophoresis 22, 421 (2001), each of which are incorporated herein by reference). These experiments monitor the number of fluorescence bursts when target molecules flow through a small detection volume. To obtain a high signal-to-noise ratio, the most common approach has been to use confocal microscopy, but the detection cross-section (about 500 nm wide×2 μm high) is much smaller than the cross-section of an ordinary microfluidic channel (100 μm×10 μm), which leads to extremely poor detection efficiency (Anal. Chem. 71, 5137 (1999), which is incorporated herein by reference). Several groups have attempted to solve this problem by decreasing the dimensions of the channel or capillary to the nanometer range so that the entire cross-section fits into the focus of the confocal microscope (Anal. Chem. 69, 3400 (1997), Electrophoresis 24, 1737 (2003), and Anal. Chem. 76, 1618 (2004), which is incorporated herein by reference). Such a small channel dimension, however, could affect electrophoretic separation of molecules in cell lysates, and also lead to clogging of the nanochannel with cell debris.

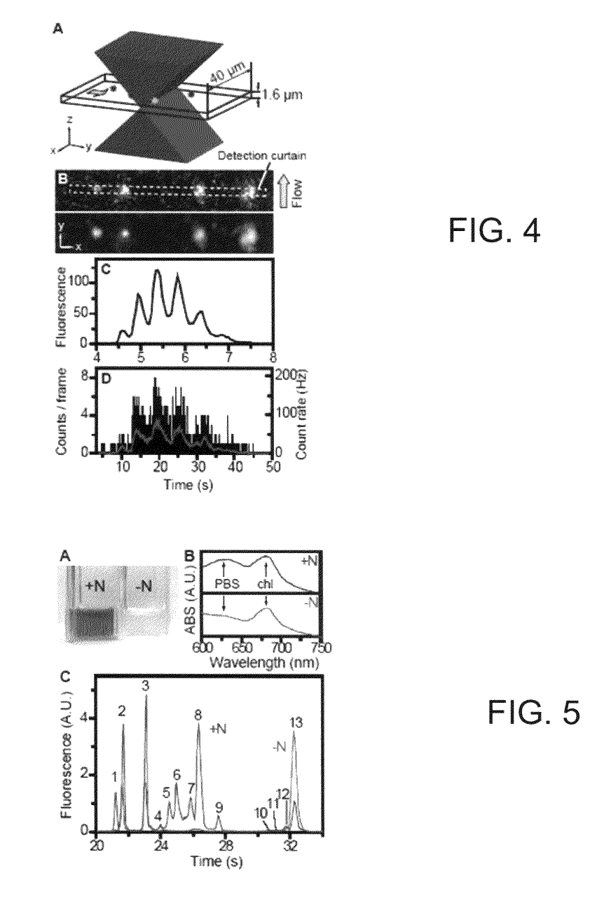

We solve the counting efficiency problem associated with confocal microscopy by widening the excitation laser focus in one direction using cylindrical optics. The excitation laser beam is focused by a cylindrical lens to form a line at the back focal plane of a high numerical aperture objective (FIG. 8A). When the laser beam emerges from the objective, it is collimated in the direction perpendicular to the channel length, thus capable of covering a channel width of tens of microns (FIG. 4A). In the other direction, the laser is still tightly focused by the objective to minimize the fluorescence background from out-of-focus excitation. The channel height needs to fit the z dimension of the excitation focus, which is about 2 μm. Using this optical configuration, the excitation laser forms a rectangular, curtain-shaped detection region across the channel. The fluorescence from molecules that pass through the curtain is recorded by an intensified CCD camera (FIG. 4B). The same excitation scheme with a photomultiplier tube as the detector can also be used for laser induced fluorescence detection. In the case where the highest detection efficiency is not required, lower numerical-aperture objectives can be used. Correspondingly, the height of the channel can be extended to about 10 μm because the laser is less tightly focused.

To characterize the molecule counting efficiency of our cylindrical optics design in capillary electrophoresis (CE), we use Alexa Fluor 647 labeled streptavidin (A647-SA) as the calibration standard. In a standard “double-T” chip made of polydimethylsiloxane (PDMS), A647-SA can be separated into multiple peaks using capillary zone electrophoresis and laser induced fluorescence detection (FIG. 4C). These peaks can be attributed to the charge ladder created when different numbers of negatively charged dyes are labeled on the streptavidin molecule (Science 272, 535 (1996), which is incorporated herein by reference). By inserting a short (10 μm long) molecule counting section into the separation channel, we resolve this charge ladder using molecule counting at a low sample concentration (FIG. 4D). A “slow-flow” method is employed to enhance the fluorescence signal for molecule counting. For molecule counting, we lower the voltages to one-tenth of the normal value when the sample peak is passing through the detection curtain. This “slow-flow” method increases the fluorescence photons collected from one molecule by increasing its dwell time in the detection curtain. At the same time, it minimizes peak broadening effects associated with extremely long migration times, which occur if a low voltage is applied in the entire separation procedure.

We find that 60% of the A647-SA molecules are counted by comparing the number of identified molecules with the number of injected molecules. Because we have suppressed the transportation loss of A647-SA molecules during separation by adding 0.1% β-D-dodecyl-N-maltoside to the separation buffer (Lab Chip 5, 1005 (2005), which is incorporated herein by reference), the lack of perfect counting is mainly caused by molecules passing through the periphery of the channel. These molecules produce lower fluorescence signals, which can be lost in the background noise. As a result, the detection efficiency varies slightly according to the brightness of a specific sample molecule. We have developed a way to estimate detection efficiencies directly from counting experiments without knowing the sample concentration (see supporting information).

As a model system for single-cell analysis, we choose to study the response of the unicellular cyanobacterium, Synechococcus sp. PCC 7942 (Synechococcus hereafter), to the depletion of nitrogen-containing nutrients in the culture medium. Cyanobacteria and some eukaryotic algae use the phycobilisome (PBS), a soluble protein-chromophore light harvesting complex, to collect the excitation energy and transfer it to the photosynthetic reaction centers. In Synechococcus cells, the PBS is mainly composed of two pigmented phycobiliproteins (PBP): phycocyanin (PC) that exists in the peripheral rods and allophycocyanin (APC) that forms the core structure. It also contains various linker polypeptides that function in assembly and in tuning the complex for efficient energy flow into the photosynthetic reaction centers. PBS attachment to photosystem II on the thylakoid membrane occurs through a chromophore-containing linker polypeptide designated L_{CM} (Ann. Microbio. (Inst. Pasteur) B134, 159 (1983) and Microbiol. Rev. 57, 725 (1993), which is incorporated herein by reference). Although isolated PC and APC molecules are highly fluorescent, they are difficult to quantify precisely in vivo by fluorescence because of the highly efficient energy transfer in the light harvesting protein complexes, their large spectra overlap, and the fluorescence background from chlorophylls in the photosystems. To detect these molecules, we lyse a single cyanobacterial cell, allow the protein complexes to dissociate, and then characterize the levels of resolved PBP complexes by capillary electrophoresis and laser induced fluorescence detection. Moreover, when grown under conditions in which certain macronutrients (such as nitrogen) are depleted, these cyanobacteria begin to degrade their PBS in an ordered way (first PC, then APC). This process reduces the absorption of excess light energy and provides cells with nutrients from the degraded PBP, helping them to attain a quiescent state in which there are almost no PBS (FIGS. 5A and 5B) (Microbiol. Rev. 57, 725 (1993) and Arch. Microbiol. 124, 39 (1980), which is incorporated herein by reference). Single-molecule detection has enabled us to analyze changes in the level and distribution of PBP complexes in individual nitrogen-starved cyanobacteria and to examine the heterogeneity of these changes among cells in a population.

Because of their cell walls, cyanobacteria are much more difficult to lyse than mammalian cells, which we have previously used for single-cell studies (P. Natl. Acad. Sci. U.S.A. 101, 12809 (2004), which is incorporated herein by reference). Traditional ways to lyse cyanobacterial cells use strong mechanical forces, such as high pressure (French press) or glass bead grinding (bead beater), both of which are difficult to integrate into a PDMS microchip design. Instead, we lyse the cyanobacteria chemically by weakening the cell walls with lysozyme and then extracting the cell contents with a reagent that contains a nonionic detergent (B-PER II from Pierce Biotech). Lysozyme treatment alone does not release pigments from a cell. On the other hand, after 2 hr or longer treatment with B-PER II, centrifugation results in colorless cell debris and a supernatant showing almost the same blue-green color as the cell suspension before lysis, indicating near complete extraction of the pigment molecules. A freeze-thaw cycle between the lysozyme and B-PER II treatments can shorten the time required for lysis to less than 1 hr by weakening the cell wall. The proteins in the cell lysate are then electrophoretically separated in a PDMS chip (FIG. 5C). We identify peaks in the electropherogram by measuring their fluorescence emission spectra and monitoring their changes when adding antibodies against different PBP and linker polypeptides. We find that most peaks represent different PBP complexes. Comparing the lysate of Synechococcus cells cultured in nitrogen-replete medium (+N) and those cultured in —N medium for more than 72 hr (−N cells), we observe that the relative intensity of peak 13 (chlorophyll a most likely of photosystem II) increases, whereas all peaks related to PC (peaks 1 and 4-9) nearly disappear. The two major PBP peaks remaining after −N growth correspond to two APC subassemblies in the PBS core (peak 2 is the APC-LcM complex and peak 3 is an APC trimer). These observations are consistent with a previously described model for chlorosis and phycobilisome degradation (Microbiol. Rev. 57, 725 (1993), which is incorporated herein by reference).

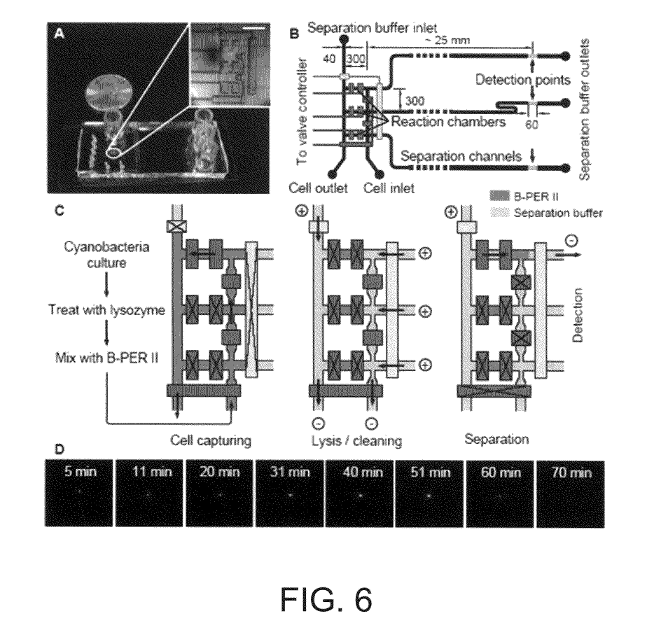

Using a PDMS single-cell analysis chip that contains a reaction chamber formed by a three-state valve and an ordinary valve, we capture and lyse a single Synechococcus cell from the culture medium. During the lysis procedure, we confirm by fluorescence microscopy that the contents of a cell are not released after lysozyme treatment. Moreover, when a lysozyme-treated Synechococcus cell is mixed with B-PER II solution, the cell contents are released in one step: after a long incubation time (usually >1 hr), PBP fluorescence from the cell drops rapidly, accompanied by the emergence of uniform fluorescence from the solution in the reaction chamber. Based on this observation, we have designed a chip with three simplified reaction chambers (FIGS. 6A and 6B) so that up to three cells can be simultaneously lysed. Our design can be easily tailored to other cells and targets. For example, by switching the reaction chamber to a three-state valve configuration, we could analyze non-fluorescent proteins from microbes or animal cells with on-chip labeling.

The analysis procedure using the present chip has three steps, which are illustrated in FIG. 6C and described in the supporting information. FIG. 6D shows a fluorescence image sequence of a Synechococcus cell. The cell fluorescence initially increases, most likely because of detachment of PBS from thylakoid membranes and their partial dissociation. This disruption of the PBS stops energy transfer to reaction centers with concomitant increased fluorescence from membrane-dissociated PBP complexes. After 50 min, fluorescence from the cell rapidly decreases, reaching a very low level after 70 min. A comparison of the cell fluorescence intensity at 50 and 70 min following exposure to B-PER II indicates the release of more than 90% of the fluorescent cell contents into the reaction chamber.

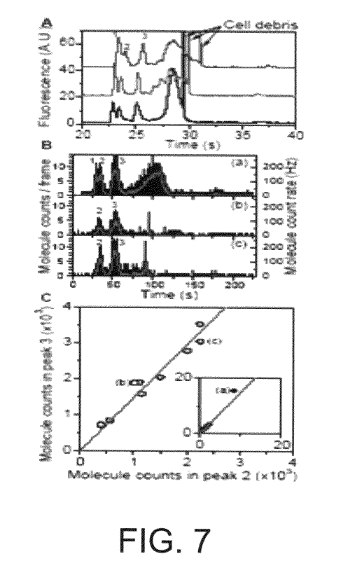

FIG. 7A shows the analysis of three +N cells in the same chip using laser induced fluorescence detection (measuring total fluorescence intensity emitted from the detection curtain). These electropherograms resemble the separation of the ensemble cell lysate in a double-T chip (FIG. 5C), although cell-to-cell variations are evident, possibly caused by genetic variation in the initial cell population used in these experiments. In another chip we analyzed ten −N cells using molecule counting to quantify the population of fluorescent complexes released following cell lysis. FIG. 7B shows three of these molecule counting results (See supporting information), and FIG. 7C shows the distribution of the molecule number of the two PBS core subassemblies (peaks 2 and 3). The molecule counts are found to have a wide distribution among the different cells. This cell-to-cell variation in overall PBP populations is much larger than that of +N cells. Interestingly, the molecule numbers of these two subassemblies show good correlation over the entire distribution range. A least square linear fitting shows that the ratio of molecule number in peak 3 to that in peak 2 is 1.5 (r²=0.93). This relationship indicates that a constant ratio of these two complexes is maintained during the degradation of the PBS under −N conditions, and that as bleaching of the cells proceeds, the complexes are simultaneously lost. These results suggest coordinated degradation of PBS components within the core of the PBS.

Among the ten −N cells examined, cell (a) in FIG. 7B is unique in that it has much brighter fluorescence and much higher molecule counts than the others. It also shows an electropherogram resembling those from +N cells, indicating an incomplete proteolysis of PBS. This cell represents about 5% of those −N cells that are atypically bright when viewed by fluorescence microscopy. In ensemble experiments, which examine cell populations, these cells would not be detected because of their low frequency of appearance. The occurrence of this rare cell is perhaps a consequence of genetic variation within the population, although more work (possibly using mutants of Synechococcus defective in phycobilisome degradation or using carefully monitored isogenic lines) needs to be done to test this hypothesis.

In conclusion, we have demonstrated that our single-cell analysis chip with single-molecule counting detection can quantify low-copy-number PBP complexes in individual Synechococcus cells. Our measurements have revealed the copy number distribution of various PBP complexes in nitrogen-starved cells and how that distribution varies among the cells in the population. These observations could not have been made using conventional methods for lysing and analyzing protein complexes in large cell populations. Analysis of the PBS assembly states during chlorosis under nitrogen-depleted conditions (or other stress conditions) could help provide a detailed map of the individual steps associated with PBS degradation and biosynthesis and the variation of these processes among individual cells.

### Supporting Information for Example 1

### Microfluidic Chip Fabrication

Polydimethylsiloxane (PDMS) microfluidic devices are fabricated in the Stanford Nanofabrication Facilities with standard soft photolithography similar to the process described previously (Proc. Nat. Acad. Sci. U.S.A. 101, 12809 (2004), which is incorporated herein by reference). The photolithography masks are designed with Freehand 10 (Macromedia) and printed on a transparency film with a high-resolution (3600 dpi) printer (Media Morphosis). To produce the silicon masters for the molecule counting chips, we first make the molecule counting section from a thin layer (˜2 μm) of negative photoresist (SU-8 2002, MicroChem). The rest of the channels are then fabricated with a 15 μm (insect cell analysis chip) or 7 μm (cyanobacteria analysis chips) layer of positive photoresist (SPR 220-7). The masters for the channel layer in valve-controlled chips are heated to 115° C. for 30 min to reflow the positive photoresist so that the channels form a smooth, round shape. The masters for the control layer of these chips are made of 40 μm thick negative photoresist (SU-8 50, MicroChem). Photoresist exposure is performed on a contact aligner (Electronic Vision 620, EV Group). The heights of the channels are measured with a surface profiler (DekTak, Veeco). The developed silicon master is treated with perfluoro-1,1,2,2-tetrahydrooctyltrichlorosilane vapor (United Chemical Technologies) in a vacuum desiccator to prevent adhesion of PDMS during the molding procedure.

The microfluidic chips are cured from PDMS prepolymer (RTV 615A and 615B, purchased from General Electric, mixed with 10:1 mass ratio) or its mixture with cyclohexane (as a thinner for spin coating). For a valve-controlled chip, the top layer (control layer) is formed by pouring mixed PDMS prepolymer on the silicon master, degassing, followed by curing at 70° C. for 30 min. After the cured PDMS piece is peeled off the master, holes are punched to connect to the pressure controller. The second layer (channel layer) is formed by spin coating a mixture of PDMS prepolymer with cyclohexane (2:1 mass ratio for insect cell analysis chips and 1:1.3 for cyanobacteria analysis chips; spin coating at 500 rpm for 18 s and then 1500 rpm for 60 s) onto the channel master and partially curing at 70° C. for 9 min. The control layer is then aligned and attached to the channel layer. More PDMS prepolymer is added to cover the silicon wafer. After curing at 70° C. for 30 min, the two layers are bonded together. The PDMS piece is peeled from the master and holes are punched to form the reagent inlets and outlets. The bottom layer is created by spin coating a mixture of PDMS prepolymer with cyclohexane (1:2 mass ratio, spin coating at 900 rpm for 9 s and then 2000 rpm for 30 s) on a microscope coverglass and curing at 70° C. for 20 min. The thickness of this PDMS layer is about 10 μm, which is required for the use of high numerical aperture objectives. The microfluidic chip is assembled by placing the PDMS piece bearing the channels on the PDMS-coated coverglass. For cyanobacteria analysis chip and “double-T” chips, short glass tubes are glued to the holes as reservoirs. The assembled PDMS chip is baked at 115° C. for 30 min to bond the channel layer to the bottom layer. “Double-T” chips that do not have the valve layer are fabricated in a similar way, without the final 115° C. baking step.

### Optical Setup and the Performance of the Cylindrical Optics:

The separation and imaging experiments are performed on a Nikon TE2000-U inverted microscope. The excitation sources are a 532-nm diode-pumped frequency-doubled Nd:YAG laser (Compass 215M, Coherent) and a 638-nm diode laser (RCL-638-25, Crystalaser), which are combined and coupled to the same single-mode optical fiber. The laser beam emerging from the optical fiber is collimated with a 100 mm achromatic lens, shaped by a 1 cm×1 cm square hole, and sent into the microscope through a spherical or cylindrical lens (each having a focal length of 400 mm). FIG. 8A shows the formation of a curtain-shaped laser focus in the microchannel by the combination of the cylindrical lens and the microscope objective. The emitted fluorescence is collected by the microscope objective and filtered by a dichroic mirror (400-535-635 TBDR, Omega Optical) and a band pass filter (HQ675/50m, Chroma). For laser induced fluorescence detection of capillary electrophoresis separation, the cylindrical lens is used for excitation, and a photon counting photomultiplier tube module (H6240-01, Hamamatsu) is used for detection, with a 50 μm slit installed at the microscope image plane to reject the out-of-focus emission. For wide-field fluorescence imaging and molecule counting, an intensified CCD camera (I-Pentamax, Roper Scientific) serves as the detector. In molecule counting experiments, the power of the laser beam emerging from the objective is about 10 mW, and the line-shaped laser focus at the sample is 50 μm long.

By imaging the fluorescence from a glass surface coated with Atto 565 labeled streptavidin (Sigma Aldrich), we can compare the z-dependence of the excitation laser strength in three different configurations: (a) wide-field, in which a spherical lens focuses the excitation laser beam to the back focal point of the microscope objective (Nikon Plan Apo 100× oil NA 1.4), (b) cylindrical, in which a cylindrical lens focuses the laser beam to the back focal plane of the objective, and (c) confocal, in which a parallel laser beam is sent into the objective. FIG. 8B shows that the confocal configuration has the sharpest drop in excitation strength when the imaging plane moves away from the focal plane, the cylindrical configuration shows similar but slightly lower z-resolution, and the wide-field configuration has almost constant excitation strength when the z position of the sample changes. A 2 μm channel fits well into the focus of the cylindrical configuration and the out-of-focus background is suppressed.

### Molecule Counting Algorithm:

When a fluorescent molecule travels across the excitation laser focus, its fluorescence is recorded by the intensified CCD camera as a bright spot in the image. We record flashes rather than tracks because the motion of the molecules through the detection curtain is faster than the time resolution of the CCD camera. During the CCD integration time (50 ms or 20 ms), multiple analyte molecules can pass the detection curtain. At a relatively low concentration, the resultant fluorescent spots are likely to appear at different locations along a line that corresponds to the position of the detection region (FIG. 4C, x direction). To identify the number of target molecules in a certain frame of the CCD image, we first use a Fourier low-pass filter to reduce the noise in the image. Continuous regions that are above a set threshold are marked. These regions are considered to be the signal from a fluorescent molecule if the following two criteria are satisfied: (1) the area of a region is larger than 15 pixels (0.76 μm²), and (2) the coordinates of the center-of-mass of a region are within the range of the detection curtain.

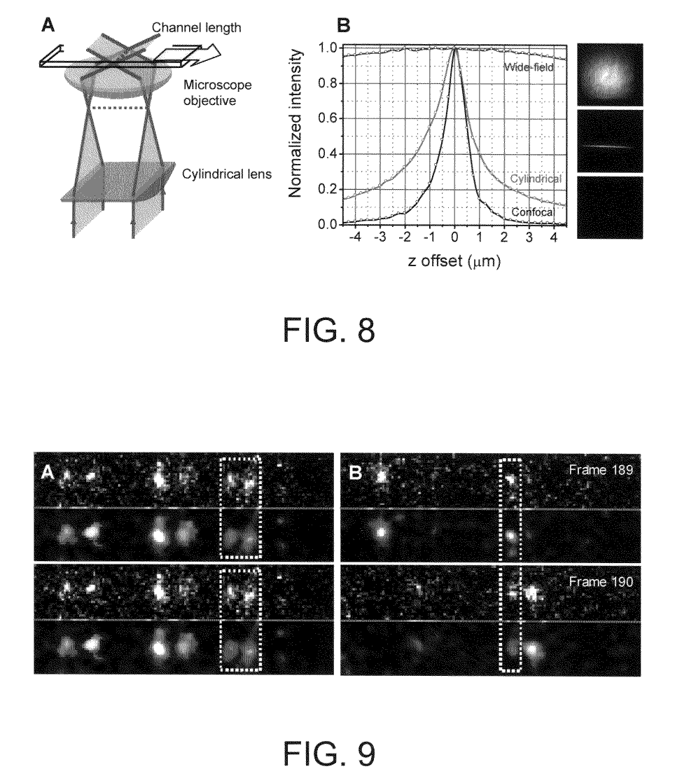

When the analyte concentration increases, more molecules are recorded in each image frame, thus increasing the probability of having two or more fluorescent spots very close together. Because each of these spots have a finite size (mainly determined by diffraction and their distance from the focal plane of the objective), when we apply the threshold, they are marked as one continuous region. Therefore, after the threshold is applied, we examine the cross-section of the image along the detection curtain (FIG. 9A). By identifying local maxima and minima in the cross-section, we can resolve closely spaced molecules.

Another source of bias in counting is the possibility that one molecule is imaged in two consecutive frames. In our slow-flow method, the time for a molecule to travel across a 1 μm wide detection region is about 2 ms; therefore, if a molecule reaches the detection region at the end of one CCD integration period, it could be recorded in the next integration period as well (the time interval between two frames is shorter than 1 ms in our intensified CCD camera). Because the Brownian motion of the molecule within this 2 ms time is not significant (comparable to the diffraction-limited laser spot size), we expect this molecule to appear at the same x positions in the two frames. Therefore, after the fluorescent spots are counted in one image frame, the x positions of their centers-of-mass are compared to those in the previous frame. If the difference is within 2 pixels (450 nm), the fluorescent spot in the second frame is marked as an invalid count (FIG. 9B).

Despite these efforts to compensate for biases in molecule counting, the chance of false negatives increases when the number of molecules in each frame is very high (>10 molecules per frame). A solution is to increase the length of the separation channel, which increases the peak width when the analyte reaches the detection point. By this means, the molecules are spread into more image frames, so that the number of molecules per frame is controlled.

### The Counting Efficiency of Alexa Fluor 647 Labeled Streptavidin:

We use a standard “double-T” microfluidic chip (see FIG. 10A) with rectangular channels to perform the molecule counting of Alexa Fluor 647 labeled streptavidin (A647-SA, purchased from Invitrogen). The concentration of A647-SA stock solution is calculated by measuring the absorbance of the protein and the dye at 280 nm and subtracting from it the contribution from the dye, determined by measuring its absorption at 647 nm. The separation buffer contains 20 mM HEPES (pH 7.5), 0.1 wt % N-dodecyl-β-D-maltoside (DDM, from AnaTrace), and 0.05 wt % sodium dodecylsulfate (SDS, from Sigma-Aldrich). The separation uses electric field strengths of about 300 V/cm. A647-SA can be separated into multiple peaks using capillary zone electrophoresis and laser induced fluorescence detection (FIG. 10C). These peaks can be attributed to the charge ladder created when different numbers of negatively charged dyes are labeled on the streptavidin molecule (Science 272, 535 (1996), which is incorporated herein by reference). By inserting a short (10 μm long) molecule counting section into the separation channel, we resolve this charge ladder using molecule counting at a low sample concentration (FIG. 10D).

We measure the size of the injection plug by imaging the injection procedure at the “double-T” junction using 200 nM A647-SA as the sample (FIG. 10B). An effective plug area is obtained by dividing the integrated intensity of the injection plug with the intensity in the channels filled with sample solution during the loading step. The injection plug volume is derived by multiplying this area by the thickness of the channel (7.6 μm). From five different measurements, we calculate that the effective size of the injection plug is 35±4 μL, which corresponds to 1557±174 injected A647-SA molecules when the sample concentration is 73 μM.

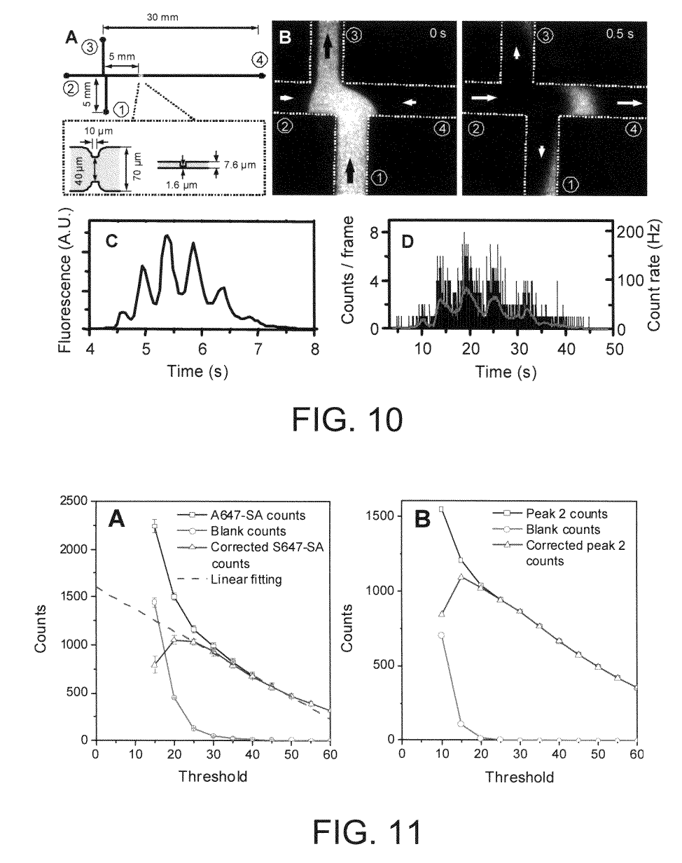

The molecule counting efficiency depends on the threshold chosen for the image analysis. A lower threshold decreases the probability of false negatives in counting but increases that of false positives from background noise. To characterize this effect, we analyze the total molecule counts from the same experiment (900 frames) with different thresholds. The molecule counts in a blank experiment (no sample is injected) are calculated in the same way. As seen in FIG. 11A, a threshold lower than 25 introduces significant false counts from background noise. Using a threshold of 30, the count from seventeen counting experiments is 929±43 molecules (after subtracting the counts from blank experiments). Therefore, the corresponding overall counting efficiency for streptavidin molecules that have different degrees of labeling is about 60%.

Two factors can contribute to the incomplete counting of sample molecules: missed molecules in identification (identification efficiency) and loss in transportation from the sample reservoir to the detection point (transportation efficiency). We measure the transportation efficiency by performing the counting experiment on a “double-T” chip that moves the detection point from 5 mm to 20 mm after the injection junction. Such experiments with the same A647-SA sample give an overall molecule counts of 961±22 (the threshold is 30, blank control is subtracted, and the difference in injected sample concentration cause by different sample loading times is corrected for), indicating that the transportation efficiency of a 15 mm channel is nearly 100% and contributes very little to the loss in counting efficiency. Therefore, we can assume that the counting efficiency is fully determined by the identification efficiency in the image analysis.

From the threshold analysis, we can actually estimate the true sample molecule counts directly. Although all molecules of the same kind have the same photophysical parameters, they show different fluorescence intensities because they are at different positions in the channel that have different excitation laser intensity. Molecules distant from the focal plane are dimmer also because their images are blurred by defocusing. A higher threshold is likely to reject more of these dim molecules. We can analyze the same set of images using different thresholds (higher than the level at which background noise starts to mix with the fluorescence signal) and interpolate the molecule counts to a threshold of zero (which hypothetically should not reject any fluorescence signal) to estimate the true molecule number. In FIG. 11A, a simple linear interpolation using the molecule counts with the threshold between 25 and 50 gives a molecule count of 1591±60, which is close to the actual number. We have found that this estimation method is applicable to the major species in our single-cell analysis (FIG. 11B). More sophisticated modeling could provide higher accuracy in estimating the true molecule counts.

### Analysis of β₂AR in SF9 Cells:

SF9 insect cells were grown at 27° C. in suspension cultures in ESF-921 medium (Expression Systems, CA) supplemented with 0.5 mg/mL gentamicin. Recombinant baculoviruses of the human β₂AR epitope-tagged at the amino-terminus with the cleavable influenza-hemagglutinin signal sequence followed by the FLAG epitope and at the carboxyl-terminus with six histidines were generated in SF9 cells using the Bac-to-Bac® Baculovirus Expression System (Invitrogen). SF9 cell cultures were infected at a density of ˜2×10⁶ cells/ml and used for experiments after 18 hr of infection.

To measure the average copy number of β₂AR by anti-FLAG M1 antibody (M1) binding, we label M1 antibody with Cy5 succinimidyl ester (GE Health Care) and purify it with a gel filtration column. The concentration of M1 is calibrated by measuring the absorption at 280 nm. 500 μL of infected SF9 cell culture is pelleted, washed with Dulbecco's phosphate-buffered saline containing Ca²⁺ and Mg²⁺ (DPBS/Ca, Invitrogen), pelleted again, and then added to 25 μL of lysis buffer containing 20 mM HEPES (pH 7.5) and 1 wt % DDM. After 10 min, 25 μL of 40 nM of Cy5-M1 in a buffer containing 20 mM HEPES (pH 7.5) and 2 mM CaCl₂ is added to the cell lysate. The binding between M1 antibody and the FLAG tag requires Ca²⁺. 10 min later, The Cy5-M1/β₂AR mixture is then separated in a “double-T” channel that has the same configuration as described previously in section 4. The separation buffer contains 20 mM HEPES (pH 7.5), 0.1 wt % DDM, 0.02 wt % SDS and 1 mM CaCl₂. Laser induced fluorescence detection is achieved using cylindrical optics and a PMT. The concentration of β₂AR is calculated by multiplying the fraction of integrated fluorescence in the β₂AR peak with total M1.

For single cell analysis, SF9 cells are harvested 18 hr after infection, washed with DPBS/Ca and adjusted to a final density of about 1 million cells per ml. The analysis using the single-cell microfluidic chip is shown in FIG. 4. Briefly, the cell suspension is injected into the chip using 3 psi of pressure. Valve 1 opens and closes until a cell is close to the three-state valve. The three-state valve then opens to introduce the cell into the reaction chamber. After the three-state valve partially closes, a low pressure is added to the air inlet through valve 2 and valve 5 to remove excess DPBS/Ca. The three-state valve fully closes before filling the channel with lysis/labeling buffer (20 mM HEPES, pH 7.5, 20 nM Cy5-M1, 1 wt % DDM, 1 mM CaCl₂) through valve 6. The three-state valve partially opens to inject the lysis/labeling buffer into the reaction chamber. Valve 2 closes to confine the volume of injection, and the reaction chamber is filled because of the air permeability of PDMS. We then fully close the three-state valve to incubate the cell with the lysis/labeling buffer for 10 min. At the same time, separation buffer (20 mM HEPES, pH 7.5, 0.1 wt % DDM, 0.02 wt % SDS, 1 mM CaCl₂) is injected through valves 3 and 7 to rinse the channels. After the lysis/labeling reaction is complete, a voltage of 1000 V is applied to the chip through valve 7, partially opened three-state valve, valve 2, and valve 4. The image acquisition starts 20 sec later and an integration time of 20 ms per frame is used. We lower the voltage to 100 V after the unreacted M1 peak passes the molecule counting section (˜46 sec after the separation starts).

### Culture of Synechococcus:

The cyanobacterium Synechococcus sp. PCC 7942 (Synechococcus hereafter) is grown in BG-11 medium (J. Phycol. 4, 1 (1969), which is incorporated herein by reference) at 30° C., illuminated at 130 μmol m⁻² s⁻¹ by incandescent bulbs, and bubbled with 3% CO₂ in air. The —N culture is deprived of nitrogen-containing nutrients in a way that is similar to the method described before (J. Bacteriol. 174, 4718 (1992), which is incorporated herein by reference). After 72 hr of nitrogen starvation, the cell culture is harvested and analyzed.

### Electrophoretic Separation of Synechococcus Lysate:

Because of their cell walls, cyanobacteria are much more difficult to lyse than mammalian cells and insect cells. Traditional ways to lyse cyanobacterial cells use strong mechanical forces, such as high pressure (French press) or glass bead grinding (bead beater), both of which are difficult to integrate into a PDMS microchip design. We have developed a method to lyse Synechococcus cells chemically. 100 to 1000 μL of Synechococcus culture is pelleted by centrifugation in a microcentrifuge and then washed with 50 μL HEPES buffer (20 mM HEPES, pH 7.5). After centrifugation, the cell pellet is mixed with 50 μL 10 mg/ml lysozyme in HEPES buffer. After 10 min of incubation at 38° C., it is washed again with 50 μL HEPES buffer and then mixed with 50 μL or 100 μL B-PER II (Pierce Biotech). Centrifugation after one hour at room temperature results in a blue-green (normal culture) or yellow (nitrogen-depleted culture) cell lysate. Because of the low ionic strength in B-PER II (20 mM Tris, pH 7.5), the phycobilisome degrades to produce smaller phycobiliprotein complexes during the lysis procedure. We have found that lysozyme treatment alone does not release pigments from a cell. On the other hand, after 2 hr or longer treatment with B-PER II, centrifugation results in colorless cell debris and a supernatant showing almost the same blue-green color as the cell suspension before lysis, indicating near complete extraction of the pigment molecules. A freeze-thaw cycle between the lysozyme and B-PER II treatments can shorten the time required for lysis to less than 1 hr by weakening the cell wall.

The cell lysate is diluted at least ten fold into a sample buffer that contains 20 mM HEPES (pH 7.5), 0.1 wt % DDM and 0.012 wt % SDS before it is added to the sample reservoir of a “double-T” chip (same dimension as shown in FIG. 10A). The other three reservoirs are filled with the separation buffer, which contains 20 mM HEPES (pH 7.5), 0.1 wt % DDM and 0.045 wt % SDS. The distance between the injection junction and the detection point is 23 mm. Continuous runs of the separation do not show significant changes in peak heights. This observation indicates that the phycobiliprotein complexes are stable in the sample buffer, but a further increase of the SDS concentration results in gradual dissociation of these protein assemblies.

The identification of the CE separation peaks is facilitated by measuring their fluorescence spectra, which are recorded by the intensified CCD camera on the same microscope. We modify the detection path by inserting a pair of relay lenses and a grating between the microscope and the camera and by placing a 50 μm wide slit at the image plane of the microscope. This modification allows the CCD camera to record wavelength information. Because phycocyanin emission overlaps with the 638 nm laser, we use the 532 nm laser as the excitation source and a dichroic mirror (565DRLPXR, Omega) and a long pass filter (565ALP, Omega) in the emission path. The transmission curves of the filters are calibrated against white light illumination, and the wavelengths in the CCD images are calibrated with the two laser lines.

By comparing the fluorescence spectra with that in the literature (Ann. Inst. Pasteur Mic. B134, 159 (1983), which is incorporated herein by reference), and by monitoring the change in the electropherogram when adding different antibodies against phycobiliproteins and linker polypeptides, we are able to identify the major peaks in the electropherogram (See Table 1). Briefly, peaks 2 and 3 are allophycocyanin complexes from the phycobilisome core; peak 6 has both allophycocyanin and phycocyanin; peaks 1, 4, 5, 7, 8, and 9 are phycocyanin complexes associated with various linker polypeptides; and peak 13 is from chlorophyll a in photosystem II.

TABLE 1
Emission maxima and identities of major peaks in the
electropherogram of Synechococcus lysate.

| | Emission | Chromophore | | Reported |
|---|---|---|---|---|
| | maximum | containing | Linker | emission |
| Peak | (nm) | protein | peptide^{a} | maximum (nm)^{b} |
| 1 | 644 | PC | L_{R}³⁰ | 643 |
| 2 | 680 | APC | L_{CM}⁷⁵ | 680 |
| 3 | 664 | APC | L_{C}^{10.5} | 662 |
| 4 | 646 | PC | Undetermined | |
| 5 | 657 | PC | Undetermined | |
| 6 | 654, 679 | 18S particle | L_{RC}²⁷ + L_{CM}⁷⁵ | 654, 680 |
| | | (S5) | | |
| 7 | 649 | PC | L_{R}33 | 648 |
| 8 | 647 | PC | None | 646 |
| 9 | 652 | PC | L_{RC}²⁷ | 652 |
| 12 | 635, 682 | phycobiliprotein | | |
| | | monomers^{c} | | |
| 13 | 679 | Chlorophyll | | |
| | | complex | | |

^{a}The denotations of the linker peptides are the same as those in (Microbiol. Rev. 57, 725 (1993), which is incorporated herein by reference).
^{b}Data from (Ann. Inst. Pasteur Mic. B134, 159 (1983)), (J. Bio. Chem. 256, 3580 (1981)) and (J. Bio. Chem. 258, 902 (1983), each of which is incorporated herein by reference).
^{c}Overlapped with peak 13.

We have also observed that emission spectra of the major peaks in the electropherogram of nitrogen-starved cell lysate matches those from normal cells, which suggests that these peaks have the same contents.

### Synechococcus Analysis Procedure:

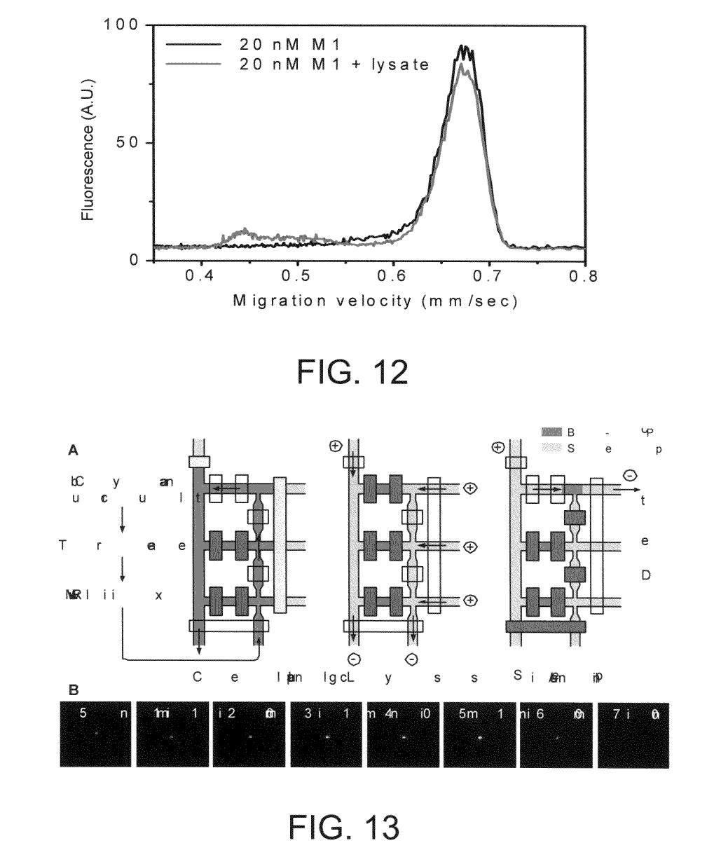

The lysis and analysis of individual Synechococcus cells is performed on a Nikon TE2000-U inverted microscope using the single-cell analysis chip having three reaction chambers (FIG. 6B). The analysis procedure has three steps (FIG. 12A):

(1) Cell capture. Synechococcus cells are treated with lysozyme, washed, diluted into B-PER II, and immediately delivered to the chip from the cell inlet. With a negative pressure applied at the cell outlet by a syringe, the cells flow through one of the reaction chambers. The valves of the reaction chamber are opened and closed randomly. At the same time, phycobiliprotein fluorescence (650 nm-700 nm) is continuously monitored by imaging through a 40× objective using wide-field illumination with the 636 nm laser. When the valve closes, if no cell or more than one cell is captured, the valve is opened to let the cell suspension continue to flow. Once an individual cell is trapped, the next reaction chamber is moved into the view field and the capturing operation is repeated. It takes less than 2 min to capture three cells after they are mixed with B-PER II; therefore, no cells are broken during the capture process.

(2) Cell lysis and chip cleaning. After capture, a fluorescent image of each cell is acquired every 10 min to monitor lysis. The excitation light is controlled by a shutter that is synchronized with the CCD acquisition, so that adverse effects (such as photobleaching) are minimized. While the cells are lysing, voltages are applied to wash out the B-PER II solution in the channels (from separation buffer inlet to cell outlet, and then from separation buffer outlets to cell inlet). After all the cells are lysed, the reservoirs are refilled with fresh separation buffer and the chip is washed again.

FIG. 12B shows a fluorescence image sequence of a Synechococcus cell. The cell fluorescence initially increases, most likely because of detachment of PBS from thylakoid membranes and their partial dissociation. This disruption of the PBS stops energy transfer to reaction centers with concomitant increased fluorescence from membrane-dissociated PBP complexes. After 50 min, fluorescence from the cell rapidly decreases, reaching a very low level after 70 min. A comparison of the cell fluorescence intensity at 50 and 70 min following exposure to B-PER II indicates the release of more than 90% of the fluorescent cell contents into the reaction chamber.

(3) Separation. To start the separation, we change the excitation path from wide-field configuration to cylindrical configuration, switch from the 40× objective to a 100×1.4 NA oil immersion objective, and move the view field to the detection point in one of the separation channels. The valves of the corresponding reaction chamber are then opened and a 1000 V separation voltage is applied simultaneously. In single molecule counting, the separation voltage is lowered to 100 V at 18.5 sec after the separation starts. The image acquisition starts at the same time when the voltage is lowered, and the integration time of the ICCD is 50 ms per frame. Cell lysate in the other two reaction chambers are analyzed sequentially.

After the separation step, the next set of cells can be introduced into the reaction chambers for re-initiation of step (1). Thus, the single-cell analysis chip can be used repeatedly, although more than 8 hr of continuous usage could cause degradation in the resolution of CE separation.

1. A single-cell analysis system, comprising:
a cell manipulation system, wherein the cell manipulation system includes a reaction chamber, a cell suspension separation system, a lysis system, and a labeling system, wherein the reaction chamber is interfaced with the cell suspension separation system, the lysis system, and the labeling system through a fluid exchange control system;
a separation system, wherein the reaction chamber is interfaced with the separation system through the fluid exchange control system; and
a detection system, wherein the detection system is interfaced with the separation system.

2. The single-cell analysis systems of claim 1, wherein the fluid exchange system is a microvalve system.

3. The single-cell analysis system of claim 1, wherein the separation system is selected from an electrophoresis system, a chromatography system, combinations thereof.

4. The single-cell analysis system of claim 3, wherein the electrophoresis system is a capillary electrophoresis system.

5. The single-cell analysis system of claim 3, wherein the chromatography system is a liquid chromatography system.

6. The single-cell analysis system of claim 1, wherein the detection system includes a detector selected from a fluorescent system, light absorbance system, and refractive index system.

7. The single-cell analysis system of claim 1, wherein the detection system includes a cylindrical fluorescence detection system.

8. The single-cell analysis system of claim 7, wherein the separation system is a capillary electrophoresis system.

9. The single-cell analysis system of claim 7, wherein the detection system quantifies the analyte is quantified by fluorescence burst counting.

10. The single-cell analysis system of claim 9, wherein the detection system includes a cylindrical fluorescence detection system.

11. The detection system of claim 10, wherein the analyte is quantified by fluorescence burst counting

12. The detection system of claim 10, wherein the analyte is quantified by measuring total fluorescence intensity.

13. A method of detecting target components in a single cell comprising:
isolating a single cell from a cell suspension including a plurality of cells;
lysing the cell to release the components in the cell;
separating the target components from the other components released from the cell; and
detecting the target components.

14. The method of claim 13, wherein the target component is selected from a target amino acids, target small molecules, target cell organelles, target polypeptide, a target polynucleotide, target polypeptide-polynucleotide complexes.

15. The method of claim 14, further comprising:
labeling the target component with a fluorescent tag to form a labeled target component prior to separating the target compounds.

16. The method of claim 15, further comprising:
separating the labeled target components from the other components that were in the cell.

17. The method of claim 16, further comprising:
detecting the labeled target component using a cylindrical fluorescence detection system as described herein.

18. The method of claim 17, wherein the detection system includes a fluorescent system.

19. The method of claim 18, wherein the detection system includes a cylindrical fluorescence detection system.

20. The method of claim 19, wherein separating is conducted using a separation system is selected from an electrophoresis system, a chromatography system, combinations thereof.

21. The method of claim 20, wherein the electrophoresis system is a capillary electrophoresis system.

22. The method of claim 20, wherein the chromatography system is a liquid chromatography system.

23. The method of claim 20, wherein isolating and lysing are conducted using a cell manipulation system, wherein the cell manipulation system includes a reaction chamber, a cell suspension separation system, a lysis system, and a labeling system, wherein the reaction chamber is interfaced with the cell suspension separation system, the lysis system, and the labeling system through the fluid exchange control system.

24. The method of claim 13, further comprising:
detecting the target compound using a cylindrical fluorescence detection system, wherein the target component is able to fluoresce without the addition of a fluorescent label.

25. The method of claim 24, wherein the detection system includes a fluorescent system.

26. The method of claim 25, wherein the detection system includes a cylindrical fluorescence detection system.

27. The method of claim 26, wherein separating is conducted using a separation system is selected from an electrophoresis system, a chromatography system, combinations thereof.

28. The method of claim 27, wherein the electrophoresis system is a capillary electrophoresis system.

29. The method of claim 27, wherein the chromatography system is a liquid chromatography system.

30. The method of claim 27, wherein isolating and lysing are conducted using a cell manipulation system, wherein the cell manipulation system includes a reaction chamber, a cell suspension separation system, a lysis system, and a labeling system, wherein the reaction chamber is interfaced with the cell suspension separation system, the lysis system, and the labeling system through the fluid exchange control system.

31. A cylindrical fluorescence detection system, comprising:
a laser system capable of emitting a laser beam; and
a cylindrical optic system, wherein cylindrical optic system is configured to receive the laser beam, wherein the cylindrical optic system includes two lenses, wherein the first lens is non-circularly symmetric with respect to the direction of the laser beam, wherein the first lens receives the laser beam, wherein the first lens is configured to focus the laser beam to form a line at a back focal plane of the second lens, wherein the first lens is configured to direct the focused laser beam to the second lens, wherein the second lens is configured to collimate the laser beam received from the first lens in the direction perpendicular to a channel length of a channel, wherein the collimated laser beam has a width that extends the width of the channel, wherein the second lens is configured to focus the laser beam received from the first lens in the direction parallel to the channel length of the channel.

32. The cylindrical fluorescence detection system of claim 31, wherein the first lens is a cylindrical lens.

33. The cylindrical fluorescence detection system of claim 32, wherein the first lens has a focal length of about 200 to 1000 mm.

34. The cylindrical fluorescence detection system of claim 31, wherein the second lens is a microscope objective.

35. The cylindrical fluorescence detection system of claim 31, wherein the width of channel is about 1 to 100 microns.

36. The cylindrical fluorescence detection system of claim 35, wherein the channel has a height of about 0.5 to 10 microns.

37. The cylindrical fluorescence detection system of claim 31, further comprising a detector selected from a CCD detector and a photomultiplier tube.

38. The cylindrical fluorescence detection system of claim 37, further comprising a slit between the sample and the detector, wherein the slit reduces the fluorescence background.
